SEQUENCE LISTING (25 sequences)

SEQ ID NO: 1 - protein, 37 aa, Artificial Sequence
  peptide
TCDDPRFQDSSSSKAPPPSLPSPSRLPGPSDTPILPQ

SEQ ID NO: 2 - protein, 38 aa, Artificial Sequence
  peptide
MTCDDPRFQDSSSSKAPPPSLPSPSRLPGPSDTPILPQ

SEQ ID NO: 3 - protein, 9 aa, Artificial Sequence
  peptide
MDDPRFQDS

SEQ ID NO: 4 - protein, 12 aa, Artificial Sequence
  peptide
RLPGPSDTPILP

SEQ ID NO: 5 - protein, 28 aa, Artificial Sequence
  peptide
DDPRFQDSILPQLLLDRLPGPSDTPILP

SEQ ID NO: 6 - protein, 20 aa, Artificial Sequence
  peptide
CTPMTRVLQGVLPALPQVVC

SEQ ID NO: 7 - DNA, 129 bp, Artificial Sequence
  dsDNA
gctagcatgacctgtgatgacccccgcttccaggactcctcttcctcaaaggcccctccccccagccttccaagcccatcccgactcccggggccctcggacaccccgatcctcccacaataagaattc

SEQ ID NO: 8 - DNA, 70 bp, Artificial Sequence
  dsDNA
gatctgtgccccaccatgacccgcgtgctgcagggggtcctgccggccctgcctcaggtggtgtgctaag

SEQ ID NO: 9 - protein, 8 aa, Artificial Sequence
  peptide
ILPQLLLD

SEQ ID NO: 10 - DNA, 25 bp, Artificial Sequence
  coding sequence
gatcctcccacaaccacccccgcct

SEQ ID NO: 11 - protein, 68 aa, Artificial Sequence [sequence omitted]

SEQ ID NO: 12 - protein, 65 aa, Artificial Sequence
  peptide
MTCDDPRFQDSSSSKAPPPSLPSPSRLPGPSDTPILPQILPQLLLDLPGGLRELQLRSLTEILKG

SEQ ID NO: 13 - protein, 66 aa, Artificial Sequence
  peptide
MTCDDPRFQDSSSSKAPPPSLPSPSRLPGPSDTPILPQILPQLLLDLFLPESFDGDPASNAPLQPE

SEQ ID NO: 14 - protein, 165 aa, Artificial Sequence
  hCG beta sub unit
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 15 - DNA, 539 bp, Artificial Sequence
  hCG beta subunit mRNA
agacaaggcaggggacgcaccaaggatggagatgttccaggggctgctgctgttgctgctgctgagcatgggcgggacatgggcatccaaggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggactcctcttcctcaaaggcccctccccccagccttccaagcccatcccgactcccggggccctcggacaccccgatcctcccacaataaaggcttctcaatccgc

SEQ ID NO: 16 - DNA, 73 bp, Artificial Sequence
  coding sequence
gatctggccgtgctagacaatggagacccgctgaacaataccacccctgtcacaggggcctccccaggataag

SEQ ID NO: 17 - DNA, 64 bp, Artificial Sequence
  coding sequence
gatctgccaggaggcctgcgggagctgcagcttcgaagcctcacagagatcttgaaaggataag

SEQ ID NO: 18 - DNA, 70 bp, Artificial Sequence
  coding sequence
gatctgtttctgccggagagctttgatggggacccagcctccaacactgccccgctccagccagagtaag

SEQ ID NO: 19 - protein, 22 aa, Artificial Sequence
  peptide
LAVLDNGDPLNNTTPVTGASPG

SEQ ID NO: 20 - protein, 19 aa, Artificial Sequence
  peptide
LPGGLRELQLRSLTEILKG

SEQ ID NO: 21 - protein, 20 aa, Artificial Sequence
  peptide
LFLPESFDGDPASNAPLQPE

SEQ ID NO: 22 - DNA, 24 bp, Artificial Sequence
  coding sequence
gatgacccccgcttccaggactcc

SEQ ID NO: 23 - DNA, 36 bp, Artificial Sequence
  coding sequence
cgactcccggggccctcggacaccccgatcctccca

SEQ ID NO: 24 - DNA, 20 bp, Artificial Sequence
  vector sequence
tccatgtcggtcctgatgct

SEQ ID NO: 25 - DNA, 7 bp, Artificial Sequence
  vector sequence
accaccc

(19) United States
(12) Patent Application Publication
(10) Pub. No.: US 2002/0025939 A1
(43) Pub. Date: Feb. 28, 2002

(54) CHORIONIC GONADORTROPIN DNA VACCINES AND METHODS

(76) Inventor: PATRICK L. IVERSEN, CORVALLIS, OR (US)

Correspondence Address: DEHLINGER & ASSOCIATES, P O BOX 60850, PALO ALTO, CA, 943060850

(21) Appl. No.: 09/466,320

(22) Filed: Dec. 17, 1999

Related U.S. Application Data
(60) Provisional application No. 60/112,910, filed on Dec. 18, 1998.

(51) Int. Cl. A61K 48/00; C07H 21/04; C12P 19/34; C12N 5/08; C12N 1/21

(52) U.S. Cl. 514/44; 435/455; 435/320.1; 435/252.33; 435/91.21; 435/375; 536/23.4; 536/23.1; 536/23.51

(57) ABSTRACT

The invention relates to immunotherapy of a mammalian subject by exposing the immune response cells of the subject to a nucleic acid construct encoding at least one hCG immunogenic epitope or precursor thereof such that the nucleic acid construct is taken up and processed by the immune response cells. The invention further relates to compositions comprising such hCG-encoding nucleic acid constructs.

Fynan et al., Proc. Natl. Acad. Sci. USA, 90:11478-11482 (1993).

Ghazal P, et al., Proc Natl Acad Sci U S A 84:3658-62 (1987).

Lan K H; et al., Gastroenterology, 111 (5):1241-51, 1996.

Lewis P J and Babiuk L A, Adv Virus Res 54:129-88, 1999.

Lietner W W et al., Vaccine 18(9-10):765-777, 1999.

Maniatis et al, (1982) Molecular Cloning: A Laboratory Manual (Cold Spring Harbor Laboratory).

Noguiez-Hellin P; et al. Proc Natl Acad Sci USA, 93(9):4175-80, 1996.

McDonnell, W. et al., N Engl J Med 334: 42-45 (1996).

Nicolet C M, et al. Cancer Gene Ther 2:161-70 (1995).

Nomura M et al., J. Immunol. Meth., 193(1)41-49, 1996.

Pardoll D M and Beckerleg A M, Immunity, 3:165-169, 1995

Pearson and Lipman, Biochem 85:2444-2448 (1988).

Pearson, Methods in Enzymol. 183:63-98 (1990).

Puhlmann M; et al., Hum Gene Ther, 10 (4):649-57, 1999.

Raz E, et al., Proc. Natl. Acad. Sci. USA 91:9519-9523 (1994).

Rivera et al., J. Cell Biol. 108:2423-2434 (1989).

Robinson, et al., Eds. The Scientific Future of DNA for Immunization, American Academy of Microbiology, 1-29 (1997).

Robinson, H L and Torres, CA, Sem. Immunol. 9:271-282 (1997a)

Roth, J. et al., J Natl Cancer Inst 89: 21-39 (1997).

Schirmbeck, et al., Eur. J. Immunol., 23, 1528-34, (1993).

Schreurs et al., Cancer Res. 58:2509-2514 (1998).

Speiser, et al., J. Immunol. 149, 972-80 (1992).

Sobol R, and Scanlon K J. Clinical protocols list. Cancer Gene Ther., 2:225-34 (1995).

Stauss, et al., Proc. Natl. Acad. Sci. U.S.A. 89, 7871-5 (1992).

Tanaka, et al., J. Immunol., 147, 3646-52 (1991).

Tang et al., Nature, 356:152-154 (1992).

Torre-Amione et al., Proc. Natl. Acad. Sci. U.S.A., 87, 1486-90 (1990).

Ulmer et al., Science, 259:1745-1749, 1993.

Wang et al., Proc. Natl. Acad. Sci. USA, 90:4156-4160, 1993.

Wang, et al., J. Immunol., 4685-4692 (1995).

Whitton et al., J. Virol. 67:(1)348-352, 1993.

Wills K N, et al. Hum Gene Ther 5:1079-88 (1994).

Wolf et al., Science 247:1465-1468 (1990).

Xiang, et al, Virology, 199:132-140, 1994.

### BACKGROUND OF THE INVENTION

Vaccination is a means for preparing the immune system to reduce disease symptoms, prevent horizontal transmission of infectious agents and reduce disease mortality. It is well known that the immune system of a subject will generate an immune response to foreign antigens. It is also known to confer immunity on an animal by administering an antibody formed elsewhere (i.e. passive immunization).

Standard vaccines include the administration of carbohydrates, peptides, polypeptides, and glycosylated polypeptides against which an immune response is desired as an “active” immunogen. An alternative to such standard immunization is the passive administration of antibodies to the subject.

Both types of “vaccination” are directed to antibody-mediated protection against an antigen of interest. However, in general a combined humoral (antibody) and cell-mediated immune response is preferred.

In addition, there are serious limitations to the use of passive immunization procedures for human therapy. These limitations are most evident in the treatment of chronic diseases such as cancer due to the cost of antibody production and the requirement for continuous administration of these antibodies. In addition, although polyclonal or monoclonal antibodies are readily produced by routine techniques, production and purification of antibody compositions which are safe for in vivo delivery is relatively expensive and time consuming.

Additional difficulties are encountered when the immunogen is a soluble protein or an endogenous protein not normally recognized by the immune system of the subject. In general, soluble proteins, e.g., proteins presented directly into the bloodstream, induce a humoral is immune response in the form of circulating antibodies. (Chen, C H and Wu, T C, 1998) In contrast, cellular immunity is generally elicited by intracellular antigens, such as antigens produced by parasites or viruses, and can also be used by the body to eliminate solid tumors. Cellular immunity involves presentation of the target antigen on the surface of antigen presenting cells in the context of MHC class I antigens. If an antigen is synthesized in a cell and presented by both Class I and Class II molecules, both antibody production and cell mediated immunity may result. (Robinson, et al., Eds., 1997).

Although induction of HLA class I restricted CTL-mediated cellular immunity by pure soluble proteins in vitro has not been reported, ex vivo induction of primary CTL are has been achieved by use of small (8 to 11-mer) synthetic peptides, which can associate with class I MHC molecules on the cell surface without the requirement for endogenous processing (Stauss, et al., Proc. Natl. Acad. Sci. U.S.A., 89, 7871-5, 1992; Carbone, et al., J. Exp. Med. 167:1767-79, 1988).

DNA vaccines have been proposed as a means to induce in vivo cellular immunity to soluble proteins and endogenous proteins not normally recognized by the immune system of the subject. For a general discussion of DNA vaccines, see for example, Pardoll D M and Beckerleg A M, Immunity, 3:165-169, 1995; Lietner W W et al., Vaccine 18(9-10):765-777, 1999; and Lewis P J and Babiuk L A, Adv Virus Res 54:129-88, 1999.

Normally, chorionic gonadotropin (CG), e.g. human chorionic gonadotropin (hCG), is secreted by cells of the human placenta and blastocyst, but not by the unfertilized ovum. However, many human cancers produce and retain and/or secrete hCG at some point during carcinogenesis. hCG composed of alpha and/or beta subunits has been detected in the membranes of a variety of human cancer cell lines (Acevedo et al., 1992). It has also been demonstrated that hCG and/or its subunits are made by the human lung cancer cells and that the hCG polypeptide or portions of it act as autocrine growth promoters for the tumor cells. (See, e.g., Rivera et al., 1989.) Additional references describe the active production of anti-hCG antibodies in tumor-bearing animals following stimulation by an hCG vaccine. (See, e.g., U.S. Pat. No. 5,762,931; and U.S. Pat. No. 4,780,312.)

Several Ohio State patents to Stevens, e.g., U.S. Pat. Nos. 4,767,842, 4,855,285 and 5,698,201, expressly incorporated by reference herein, disclose the use of a beta-hCG/tetnus toxoid modified peptide as an anti-cancer strategy based on antibody production against hCG by the host.

However, direct vaccination with intact, soluble, chorionic gonadotropin antigens is most likely to result in entry into the Class II MHC pathway of antigen presentation and to result in a CD4+ helper T cell-mediated immune response and not a CD8+ cytotoxic T cell-mediated cellular immune response. In contrast, when exposed to the immune response cells of a subject, a CG-encoding DNA vaccine should be taken up by the cells, the encoded antigens translated in the cytoplasm, such that it can enter the major histocompatibility complex (MHC) class I pathway. Only proteins that originate inside a cell are processed in this manner. (McDonnell, W. et al., N Engl J Med 334: 42-45, 1996).

There remains a need for a safe and effective method of reducing or eliminating the level of circulating CG and cell-associated CG in cancer patients with CG-expressing tumors.

An additional and well known biological activity of CG is its association with fertility. Accordingly, a safe and effective method of producing an immune response to CG would also be useful in fertility control.

### SUMMARY OF THE INVENTION

The present invention provides methods for immunotherapy of cancer which cancer expresses human chorionic gonadotropin (hCG), or a subunit thereof.

The present invention further provides methods for fertility control based on inducing an immune response to chorionic gonadotropin (hCG), or a subunit thereof.

The present invention addresses one or more of the drawbacks inherent in the prior art by providing novel methods for generating a multivalent immune response against immunogenic epitopes of an endogenous protein, hCG, in vivo in a subject, as an effective method of immunotherapy.

In one aspect, the invention relates to hCG-encoding nucleic acid constructs (DNA vaccines) and methods of eliciting an immune response against hCG by exposure of the immune response cells of a mammalian subject, particularly a human subject, to such nucleic acid constructs, as a means to diminish, prevent the spread of, and/or progression of cancer.

The “immune response” conferred by the methods of the invention can be a humoral (antibody) and/or a cell mediated immune response to one or more immunogenic epitopes of hCG, but more importantly interferes with the progression, spread, and/or growth of a tumor or other malignancy.

In most cases, administration of an hCG-encoding nucleic acid construct (DNA vaccine) to a patient is expected to elicit both a humoral and a cell mediated immune by the subject's immune system.

In one aspect, administration of a DNA vaccine of the present invention is effective to result in an antigen-specific cytotoxic T-lymphocytes (CTL)-mediated response against major histocompatibility complex class I (MHC-I) restricted hCG peptides.

In another aspect, the DNA vaccines of the present invention are effective to elicit a T cell mediated humoral immune response resulting in production of antibodies by the subject which are directed against one or more hCG immunogenic epitopes.

In a further aspect the DNA vaccines of the present invention are effective to elicit both a T helper 1 (Tₕ₁) and T helper 2 (Tₕ₂) type of T cell response by the subject.

The invention further provides methods of administering a DNA vaccine to a subject comprising a recombinant DNA expression vector which includes the coding sequence for an hCG immunogenic peptide, polypeptide or precursor thereof, operably linked to control sequences necessary for expression in vivo in the subject.

In a further aspect, the invention provides vectors for use in such DNA vaccination methods and constructs comprising such DNA vaccines for use in immunotherapy, e.g., immunotherapy of cancer.

The desired hCG antigen encoded by the DNA vaccine is an immunogenic epitope or a precursor of an immunogenic epitope of hCG which is hCG-specific, i.e., it does not cross react with other related compounds, such as leutinizing hormone (LH) or follicle stimulating hormone (FSH).

The DNA vaccine can be monovalent or polyvalent and accordingly encode one or more hCG immunogenic epitopes. When polyvalent, the DNA vaccine preferably encodes at least two hCG immunogenic epitopes, one or more of which may be a precursor of an hCG immunogenic peptide capable of being processed by the subject to more than one hCG immunogenic epitope.

In one aspect, the DNA vaccines of the present invention encode a fusion polypeptide or protein that has a first sequence which encodes an hCG immunogenic peptide or precursor thereof in reading frame alignment with a second sequence which encodes a cytokine or immune system stimulator, such that administration of the DNA vaccine results in co-expression of the hCG immunogenic peptide together with the cytokine or immune system stimulator in the same host cell.

In an additional aspect, the invention provides methods for eliciting an immune response to a number of hCG immunogenic epitopes by administering to a subject, an expression vector comprising fragments of a library derived from the nucleotide sequence which encodes the beta subunit of hCG. The administration of a DNA vaccine comprising an hCG beta subunit expression library allows for identification of immunogenic epitopes by an evaluation of the immune response of the subject against hCG peptides following such administration.

In a further aspect, the invention provides for the use of CG-encoding DNA vaccines in fertility control in a mammal. Methods include exposure of the immune response cells of a subject to CG-encoding nucleic acid constructs which results in an immune response by the subject that neutralizes a biological activity of CG associated with fertility.

These and other objects and features of the invention will be more fully appreciated when the following detailed description of the invention is read in conjunction with the accompanying drawings.

### BRIEF DESCRIPTION OF THE FIGURES

FIG. 1A depicts the amino acid sequence of the beta subunit of human chorionic gonadotropin (hCG) as provided in GenBank Accession number 180437, designated SEQ ID NO:14.

FIG. 1B depicts residues 109-145 of the beta subunit of hCG (C-terminal peptide, CTP), designated SEQ ID NO:1.

FIG. 1C depicts the CTP (SEQ ID NO:1), with an added N-terminal methionine, designated SEQ ID NO:2.

FIG. 1D depicts residues 111-118 (epitope #1) of the amino acid sequence of the beta subunit of hCG, designated SEQ ID NO:3.

FIG. 1E depicts residues 133-144 (epitope #2) of the amino acid sequence of the beta subunit of hCG, designated SEQ ID NO:4.

FIG. 1F depicts the amino acid sequence of a linker peptide, designated SEQ ID NO:9.

FIG. 1G depicts the amino acid sequence of a fusion protein comprising epitopes #1 and #2 of the CTP of the beta subunit of hCG with an intervening linker sequence (SEQ ID NO:9), designated SEQ ID NO:5.

FIG. 1H depicts the amino acid sequence of residues 38-57 of the beta subunit of hCG with a disulfide bond linking residues 38 and 57 (the “loop” peptide), designated SEQ ID NO:6.

FIG. 1I depicts the sequence of a fusion protein comprising the CTP 38-mer (SEQ ID NO:2), and amino acid residues 115-136 of the Her-2 protein (SEQ ID NO:19) with an intervening linker sequence (SEQ ID NO:9), designated SEQ ID NO:11.

FIG. 1J depicts the sequence of a fusion protein comprising the CTP 38-mer (SEQ ID NO:2), and amino acid residues 134-151 of the Her-2 protein (SEQ ID NO:20) with an intervening linker sequence (SEQ ID NO:9), designated SEQ ID NO:12.

FIG. 1K depicts the sequence of a fusion protein comprising the CTP 38-mer (SEQ ID NO:2), and amino acid residues 376-395 of the Her-2 protein (SEQ ID NO:21) with an intervening linker sequence (SEQ ID NO:9), designated SEQ ID NO:13.

FIG. 1L depicts the amino acid sequence of residues 115-136 of the Her-2 protein, designated SEQ ID NO:19.

FIG. 1M depicts the amino acid sequence of residues 134-151 of the Her-2 protein, designated SEQ ID NO:20.

FIG. 1N depicts the amino acid sequence of residues 376-395 of the Her-2 protein, designated SEQ ID NO:21.

FIG. 2A depicts the nucleic acid sequence which encodes the beta subunit of human chorionic gonadotropin as provided in GenBank Accession numbers J00117, M38559 and M54963, designated SEQ ID NO:15. The coding sequence for epitope #1 of the beta subunit of hCG (SEQ ID NO:22) is bolded and the coding sequence for epitope #2 (SEQ ID NO:23) is italicized in the figure.

FIG. 2B depicts the nucleic acid sequence which encodes the CTP 38-mer of the beta subunit of hCG (SEQ ID NO:2), designated SEQ ID NO:7. The upper case letters represent the nucleic acid from the vector and the lower case letters represent the nucleic acid sequence of the insert produced by DNA synthesis. A single letter amino acid sequence is provided below the nucleic acid sequence.

FIG. 2C depicts the nucleic acid sequence which encodes residues 38-57 of the beta subunit of hCG (SEQ ID NO:6), designated SEQ ID NO:8.

FIG. 2D depicts the nucleic acid sequence which encodes the linker peptide, (SEQ ID NO:9), designated SEQ ID NO:10.

FIG. 2E depicts the nucleic acid sequence which encodes the fusion protein (SEQ ID NO:11) comprising amino acid residues 115-136 of the Her-2 protein, designated SEQ ID NO:16.

FIG. 2F depicts the nucleic acid sequence which encodes the fusion protein (SEQ ID NO:12) comprising amino acid residues 134-151 of the Her-2 protein, designated SEQ ID NO:17.

FIG. 2G depicts the nucleic acid sequence which encodes the fusion protein (SEQ ID NO:13) comprising amino acid residues 376-395 of the Her-2 protein, designated SEQ ID NO:18.

FIG. 3 is a diagrammatic illustration of the plasmid pCl-neo.

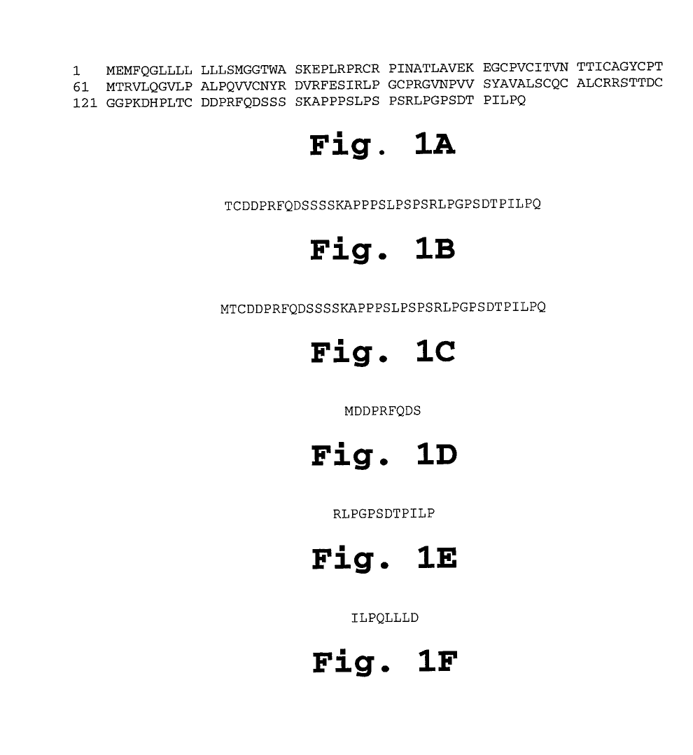

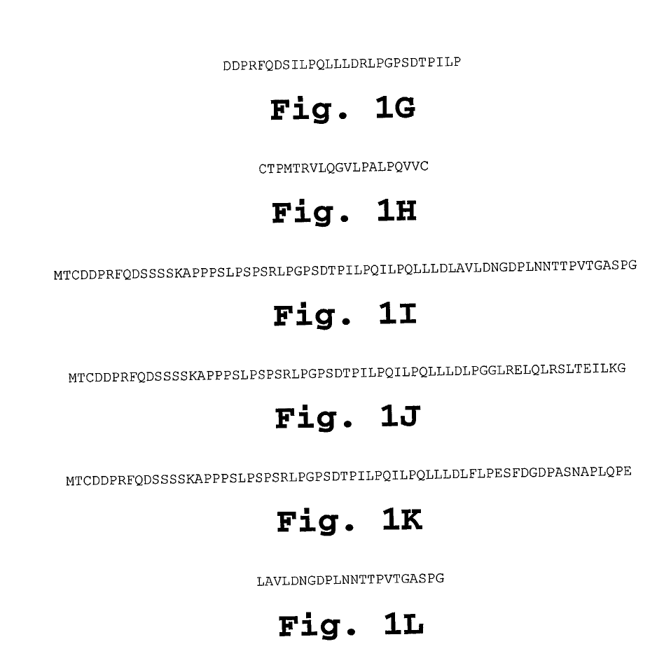

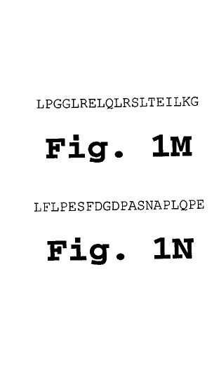

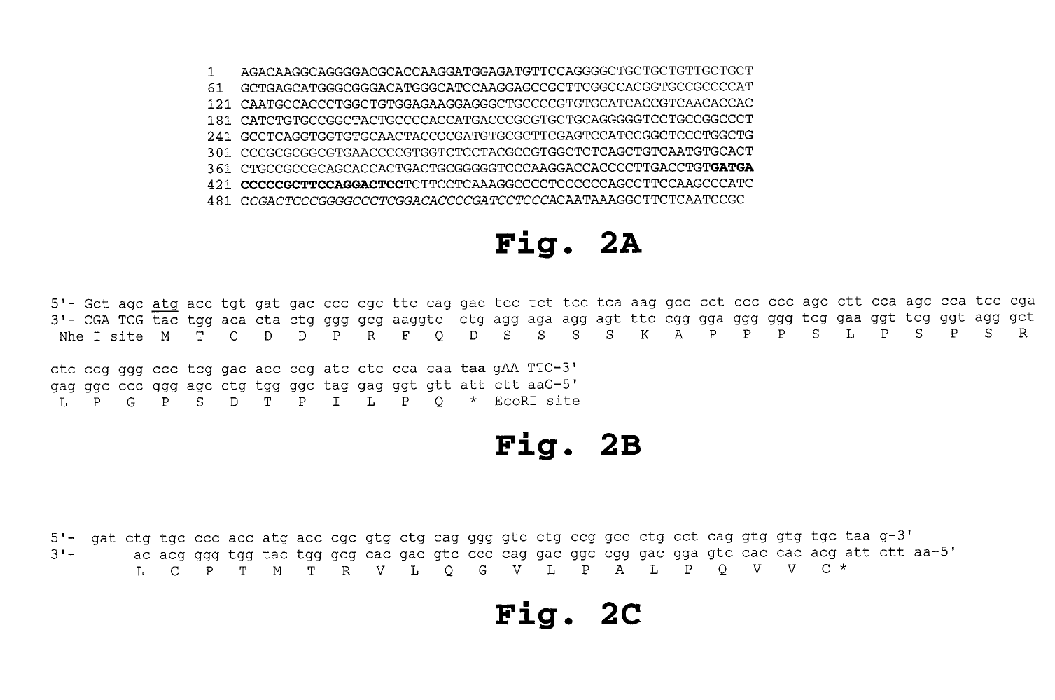

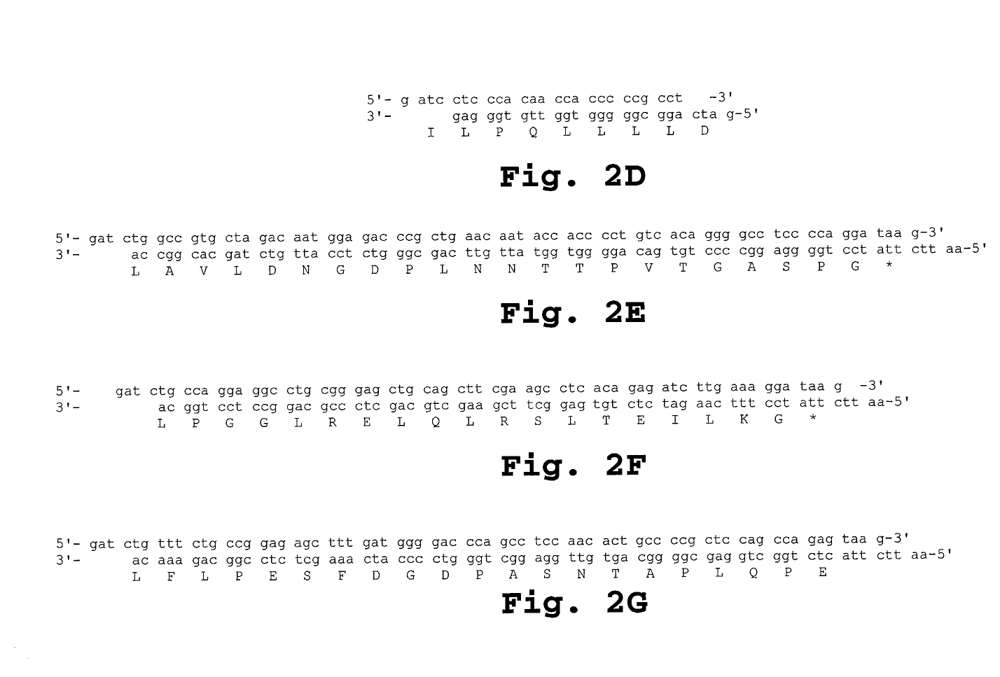

### DETAILED DESCRIPTION OF THE INVENTION

I. Definitions

The term “nucleic acid construct”, as used herein is used interchangeably with the term “DNA vaccine” and includes DNA or RNA which encodes (1) the full length hCG amino acid sequence, (2) the hCG beta subunit amino acid sequence, or (3) one or more immunogenic epitopes of hCG comprising peptide antigens having at least 8 amino acids. It will be understood that the DNA vaccine-encoded amino acid sequences of (1) and (2) are capable of being processed to immunogenic hCG peptides of from about 8 to 40 amino acids in length.

The term “hCG peptide” and “hCG epitope” refer to an amino acid sequence which is the same as part of but not all of the amino acid sequence of the entire hCG protein, and which retains at least one biological function or activity of the entire hCG protein, for example, a fragment which retains an immunological activity of the full hCG protein.

The term “hCG immunogenic polypeptide” or “hCG immunogenic beta subunit polypeptide” or fragments thereof as used herein refer to amino acid sequences derived from hCG or the beta subunit of hCG, respectively, which are capable of eliciting a cellular and/or humoral immune response when exposed to the immune response cells of an immunocompetent subject. Such an immune response may require antigen processing in conjunction with class I and/or II major histocompatability antigens (MHC).

The terms “antigenic precursor” or “precursor” relative to hCG immunogenic epitopes, as used herein refer to hCG peptides capable of being processed to hCG immunogenic peptides by the cells of the subject.

The term “hCG C-terminal peptide” or “hCG CTP” as used herein refer to the C-terminal 37 amino acids of the beta subunit of hCG (“CTP 37-mer”). In some cases, the hCG CTP has an added methionine at the N-terminus (termed the “CTP 38-mer”).

The term “loop peptide”, as used herein with reference to hCG means amino acids 38 to 57 of the beta subunit of hCG wherein amino acids 38 and 57 are linked by a disulfide bridge. The “loop peptide” may be presented as the native sequence or a variant thereof.

The term “non-native” as used herein relative to an immunogenic hCG epitope, means the amino acid sequence of the epitope differs, by one or more amino acids from the amino acid sequence of the same hCG immunogenic epitope, as it is found in nature. The “non-native” amino acid sequence may comprise an hCG immunogenic epitope having a variant amino acid sequence which contains one or more “conservative” or “non-conservative” amino acid substitutions, insertions or deletions.

The term “non-native” as used herein relative to the coding sequence for an immunogenic hCG epitope, means the nucleic acid which encodes the epitope differs by one or more nucleotides from the coding sequence for the same hCG immunogenic epitope as it is found in nature. The “non-native” nucleic acid sequence generally encodes an hCG immunogenic epitope having a variant amino acid sequence which contains one or more “conservative” or “non-conservative” amino acid substitutions, insertions or deletions. Furthermore, a “non-native” nucleic acid sequence may encode an hCG immunogenic epitope having the same amino acid sequence as the native hCG epitope but, due to the degeneracy of the genetic code, have a nucleic acid sequence which is altered by one or more nucleotides from the reference polynucleotide sequence.

The terms “Class II major histocompatability complex”, “Class II MHC” and “Class II”, as used herein refer to molecules that are expressed on various cell types and which play an essential role in the recognition of protein antigens by T cells. Class II MHC molecules typically bind peptides of from about 7 to 30 or more amino acids and form complexes that are recognized by antigen-specific CD4+ T cells. Such peptide/CD4+ T cell complexes facilitate antibody production against the peptide antigen by an immunocompetent subject.

The term “immune response” as used herein refers to a cellular immune response such as a cytotoxic T cell response and/or a humoral immune response such as production of antibodies against an immunogenic epitope.

The term “immunocompetent subject”, as used herein refers to a subject having immune response cells which upon exposure to an immunogenic epitope, is capable of mounting a cellular and/or humoral immune response against the immunogenic epitope. The invention is useful for both the human and other mammalian subjects.

The term immunogenic “epitope” or “antigenic determinant”, as used herein refers to a portion of the hCG amino acid sequence which will generate a T- and/or B-cell mediated immune response against hCG. It is preferred that the epitope be unique; that is, an immune response generated to the specific hCG epitope show little or no cross-reactivity with other antigens.

The term “active immunization”, as used herein means the administration of a vaccine which induces an immune response by the immune response cells of the subject. “Active immunization” may be achieved by exposure of the immune response cells of the subject to a nucleic acid sequence or an amino acid sequence.

The term “exposing”, as used herein means bringing the immune response cells of the subject in contact with a nucleic acid construct. Such “exposing”, may take place in vitro, e.g., by introduction of the construct into a host cell by calcium phosphate transfection, DEAE-Dextran mediated transfection, or electroporation, (Davis, L., Dibner, M., and Battey, I. BASIC METHODS IN MOLECULAR BIOLOGY, 1986), or in vivo, e.g., by introduction of a “naked” nucleic acid construct into a host by injection into muscle or other tissue (Wolf et al., 1990).

The term “passive immunization”, as used herein is meant the direct administration of antibodies to a subject, as an immunization approach.

The term “immune response cells”, as used herein refers to the cells of a subject which are capable of processing antigens and presenting them in conjunction with Class I or Class II MHC.

The term “polynucleotide” as used herein refers to a polymeric molecule having a backbone which supports bases capable of hydrogen bonding to typical polynucleotides, where the polymer backbone presents the bases linked by phosphodiester bonds in a manner to permit such hydrogen bonding in a sequence specific fashion between the polymeric molecule and a typical polynucleotide (e.g., single-stranded DNA). “Polynucleotides” include polymers having modifications, e.g., those involving phosphodiamidate morpholine (PMO) chemistry.

The term “recombinant nucleic acid”, as used herein refers to a nucleic acid sequence originally formed in vitro, generally by the manipulation of the nucleic acid by endonucleases, in a form not normally found in nature.

A “heterologous” nucleic acid coding sequence is a structural coding sequence that is not native to the cell being transformed, or a coding sequence that has been engineered for improved characteristics of its protein product. Such a “heterologous” coding sequence is not normally found contiguous to, or associated with, the promoter with which it is used, e.g., a CMV immediate early promoter/enhancer adjacent a nucleic acid sequence encoding an hCG immunogenic epitope.

Nucleic acid subunits are referred to herein by their standard base designations; T, thymine; A, adenosine; C, cytosine; G, guanine, U, uracil; variable positions are referred to by standard IUPAC abbreviations: W, A or T/U; R, A or G; S, C or G; K: G or T/U (37 CFR. §1.822).

The nucleic acid may be double stranded, single stranded, or contain portions of both double stranded or single stranded sequence. The depiction of a single strand also defines the sequence of the other strand and thus also includes the complement of the sequence.

The term “expression vector”, as used herein refers to a nucleic acid construct containing a nucleic acid sequence which is operably linked to a suitable control sequence capable of effecting the expression of said nucleic acid in a suitable subject. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable subject, the vector may replicate and function independently of the subject's genome, or may, in some instances, integrate into the genome itself. In the present specification, “plasmid” and “vector” are sometimes used interchangeably as a DNA plasmid vector is the most commonly used form of vector at present. However, the invention includes such other forms of expression vectors which serve equivalent functions and which are, or become, known in the art, e.g. an mRNA transcript.

The term “operably linked”, as used herein when describing the relationship between two nucleic acid or polypeptide regions simply means that they are functionally related to each other. For example, a pre-sequence is operably linked to a peptide if it functions as a signal sequence, participating in the secretion of the mature form of the protein most probably involving cleavage of the signal sequence. A promoter is operably linked to a coding sequence if it controls the transcription of the sequence; a ribosome binding site is operably linked to a coding sequence if it is positioned so as to permit translation.

The term “promoter sequence” as used herein refers to the minimal sequence sufficient to direct transcription. Also included in the invention is an enhancer sequence which may or may not be contiguous with the promoter sequence. Enhancer sequences influence promoter-dependent gene expression and may be located in the 5′ or 3′ regions of the native gene. Optionally, expression is cell-type specific, tissue-specific, or species specific.

A “regulatable promoter” is any promoter whose activity is affected by a cis or trans acting factor (e.g., an inducible promoter, such as an external signal or agent).

“Constitutive promoter” is any promoter that directs RNA production in many or all tissues of a plant transformant at most times, e.g., the human CMV immediate early enhancer/promoter region which promotes constitutive expression of cloned DNA inserts in mammalian cells.

The term “homology” or “homologue” as used herein refers to the level of identity between two sequences, i.e., 70% homology means the same thing as 70% sequence identity when determined by the algorithms described below, and accordingly a homologue of a given sequence has at least about 70% or 80%, preferably about 80%, 85%, 90% or 95% sequence identity over a given length of the sequence.

“Nucleic acid sequence identity” is determined essentially as follows. Two polynucleotide sequences of the same length are considered to be identical to one another, if, when they are aligned using the LALIGN program, over 60%, preferably about 70%, preferably about 80%, more preferably about 85%, even more preferably about 90% sequence are determined to be identical when aligned using the default parameters and the default PAM matrix. The LALIGN program is found in the FASTA version 1.7 suite of sequence comparison programs (Pearson and Lipman, 1988; Pearson, 1990; program available from William R. Pearson, Department of Biological Chemistry, Box 440, Jordan Hall, Charlottesville, Va.). Alternatively, the BLAST2 version of the BLASTN comparison program found at http://www.ncbi.nlm.nih. gov/BLAST/ on the internet, which allows evaluation of sequence identity with GenBank database sequence entries using default parameters and the default BLOSUM 62 matrix, may be used.

Amino acid residues are referred to herein by their standard single letter notations: A, alanine; C, cysteine; D, aspartic acid; E, glutamic acid; F, phenylalanine; G, glycine; H, histidine; I, isoleucine; K, lysine; L, leucine; M, methionine; N, asparagine; P, proline; Q, glutamine; R, arginine; S, serine; T, threonine; V, valine; W, tryptophan; Y, tyrosine.

“Amino acid sequence identity” with respect to the amino acid sequences identified herein is defined as the percentage of amino acid residues in a candidate sequence that are identical with the amino acid residues in the native sequence, after aligning the sequences and introducing gaps, if necessary, to achieve the maximum percent sequence identity, and not considering any conservative substitutions as part of the sequence identity. Such amino acid sequence identity may be determined using the ALIGN program or the BLASTP comparison program found at http://www.ncbi.nlm.nih.gov/BLAST/ on the internet, with default parameters, as described above.

By “functional equivalent” is meant a nucleic acid sequence which encodes an hCG peptide or peptide having equivalent pharmacological activity. The parts or residues constituting the active region of the compound are known as the “pharmacophore”. Equivalent pharmacophores will have an identical pharmacological activity.

Two nucleic acid fragments are considered to be “selectively hybridizable” to a reference polynucleotide if they are capable of specifically hybridizing to the polynucleotide or variants thereof or of specifically priming a polymerase chain amplification reaction: (i) under moderate or high stringency hybridization and wash conditions, as described, for example, in Maniatis, et al. (1982), pages 320-328, and 382-389; (ii) under reduced stringency wash conditions that allow at most about 25-30% base pair mismatches, for example:2×SSC (contains sodium 3.0 M NaCl and 0.3 M sodium citrate, at pH 7.0), 0.1% sodium dodecyl sulfate (SDS) solution, room temperature twice, 30 minutes each; then 2×SSC, 0.1% SDS, 37° C., once, for 30 minutes; then 2×SSC, at room temperature twice, for 10 minutes each; or (iii) under standard PCR priming and anmplification conditions (for example, as described in Saiki, et al., 1988), which result in specific amplification of sequences of the desired target sequence or its variants.

The term “vaccinate”, as used herein relative to a DNA vaccine means administration of a nucleic acid construct encoding one or more hCG amino acid sequences to a subject with the purpose of inducing an immune response to one or more immunogenic epitopes of hCG.

As used herein, the term “cancer” refers to a cell that exhibits a loss of growth control and forms unusually large clones of cells. Cancer cells generally have lost contact inhibition and may be invasive and/or have the ability to metastasize.

The term “treating cancer”, as used herein means any intervention used in an attempt to alter the natural course of a cancer. Treatment includes, but is not limited to, administration of a cellular composition, chemotherapeutic agent and/or other treatment to a subject diagnosed as having cancer, with the intention of slowing or eliminating the growth of cancer cells or a solid tumor.

The term “fertility control”, as used herein refers to prevention of pregnancy in a mammal.

II. Cancer, Fertility and hCG

Although malignant tumors may express protein antigens that are recognized as foreign by the subject, and immune surveillance may limit the growth and spread of some types of tumors, the immune system does not effectively protect the subject from lethal human cancers. Such tumors may overwhelm the immune system due to rapid growth and spread and/or the tumor cells may evade immune destruction. Although the mechanism is not part of the present invention, proposed mechanisms for such evasion include, (1) down-regulation of Class I MHC antigens on the surface of tumor cells resulting in little or no complexing of processed tumor peptide antigens with Class I MHC as required for recognition by cytotoxic T lymphocytes (CTL), (2) a lack of activation of CTL due to little or no expression of Class II MHC molecules by tumor cells such that they cannot directly activate tumor-specific CD4+ helper T cells (which produce signals likely to be needed for CTL activity), (3) a lack of co-stimulation cell surface markers that provide secondary signals for activation of CD4+ helper T cells, and (4) factors produced by tumor cells that suppress anti-tumor responses, such as fas-ligand. [Abbas, AK et al., Eds., CELLULAR AND MOLECULAR IMMUNOLOGY, 3^{rd} edition, WB Saunders Co., 394-405 (1997)]

Investigators have also determined, that certain polypeptides are supportive factors for and/or secreted by, neoplastic cells in both man and other mammals. Such supportive factors have biochemical, biological and immunological similarity to hormones, particularly chorionic gonadotropin (CG). Certain carcinomas exude CG or an immunologically-similar material on their surfaces, thereby presenting to the immune system of the subject a surface which appears to be formed of material endogenous to the subject and which is thus relatively non-immunogenic.

An effective treatment of malignant cancers must prevent further spread of neoplastic cells and reduce mortality from the disease. Current treatment methods including non-specific stimulation of the immune system, passive immunotherapy, and active immunization, for example, vaccination with killed tumor cells or tumor cell antigens, polypeptides or fragments thereof.

Active immunization studies have indicated that a beta-hCG/tetanus toxoid modified polypeptide confers upon rats protection against an injection of tumor cells of the virulent rat mammary adenocarcinoma R 3230 AC, which is associated with CG-like material. Passive immunization has also resulted in protection against Lewis lung carcinoma tumors, viral-induced leukemia and a sarcoma tumor in mice. (See, e.g., U.S. Pat. No. 5,698,201.)

Chorionic Gonadotropin (CG) is the “pregnancy hormone” that keeps the corpus luteum producing progesterone when conception occurs. It has been shown that antibodies to CG are capable of neutralizing the biological activity of endogenous CG.

Clinical trials using vaccines containing human beta hCG chemically linked to tetanus toxoid, have resulted in production of antibodies against human beta hCG and prevention of pregnancy. See, e.g., Talwar G P et al., Am J Reprod Immunol 37(2):153-60, 1997; Schutze M P et al., Am J Reprod Immunol Microbiol 14(3):84-90, 1987.

CG DNA vaccines find utility in fertility control in both humans and animals. Such fertility control may be used as a means to temporarily interfere with reproduction or may be used for long term or continuous fertility control.

III. Immunotherapy with DNA vaccines

Some of the disadvantages of conventional vaccines may be overcome by using what is called “genetic immunization” by administration of DNA vaccines (Tang et al., 1992). This technology involves inoculating a simple, nucleic acid construct encoding a polypeptide or peptide into the cells of the host (Fynan, et al., 1993; Ulmer, et al, 1993).

The production of DNA vaccines is straightforward and nucleic acid constructs have been shown to be more stable than proteinaceous vaccines. Genetic immunization with DNA vaccines containing specific sequences has shown promise in several model systems. Exemplary applications include: Schreurs et al., 1998, cellular and humoral immunity against human melanocyte differentiation antigen; Davis, et al, 1993, hepatitis B surface antigen; Conry, et al, 1994, carcinoembryonic antigen; Xiang, et al, 1994, rabies virus glycoprotein; Cox, et al, 1993, Bovine Herpesvirus; Ulmer et al., 1993, influenza A; and Johnston, SA, et al., U.S. Pat. No. 5,703,057, mycoplasma.

Thus, the use of nucleic acid constructs comprising DNA or RNA have the potential to overcome some of the problems encountered when a host is presented directly with an antigen by facilitating: (1) prolonged in vivo antigen expression resulting in long-lasting cellular and humoral immunity; (2) involvement of antigen presenting cells; and (3) the ability to generate an immune response to multiple epitopes derived from the encoded antigen(s) (Schreurs et al., 1998; Felgner, 1998). In addition, DNA vaccines are more stable and easier to manipulate than intact proteins (Felgner, 1998). See also, McDonnell, W. Michael et al., N Engl J Med 334: 42-45 (1996), which discloses that one year after vaccination with an influenza DNA vaccine, mice remain fully protected against a lethal dose of homologous influenza.

Recently, this technology has been applied to cancer for the generation of cancer vaccines, and the related studies have resulted in at least three clinical protocols for the generation of anti-tumor immunity against colon cancer and melanoma (Roth, J. et al., J Natl Cancer Inst 89: 21-39, 1997; Sobol R, and Scanlon K J. Clinical protocols list. Cancer Gene Ther.,2:225-34, 1995). However, this method of delivery is limited to cells near the injection site capable of acquiring the DNA, and lacks tissue targeting. The delivery of DNA by particle bombardment can generate gene expression in the liver and in tumors, but also suffers from a lack of targeting and requires a surgical procedure to allow access to the tissue (Nicolet CM, et al. Cancer Gene Ther 2:161-70, 1995).

The present invention provides CG-encoding nucleic acid constructs for exposure to the immune response cells of a subject such that an immune response to CG is mounted to various antigenic CG epitopes. Accordingly, the exposure of a DNA vaccine can be effective to neutralize the role of CG polypeptides or fragments thereof in facilitating survival and/or growth of a tumor or malignancy. For example, tumors in a human cancer patient may be treated by immunization with a DNA vaccine such that the host develops an immune response specific to human CG or another analogous factor which are facilitating survival and/or growth of the malignancy.

In most cases, Class I molecules present foreign proteins synthesized in a cell. For presentation by Class II, the foreign protein either can be synthesized in the cell or taken up by the cell from the outside (i.e., presented in the form of a free protein or peptide). If an antigen is synthesized in a cell and presented by both Class I and Class II molecules, both antibody producing B cells and cytotoxic T cells are produced. However, if an antigen originated outside of a cell and is expressed only by Class II, the specific immune response is largely limited to T helper cells and antibody production (THE SCIENTIFIC FUTURE OF DNA FOR IMMUNIZATION, American Academy of Microbiology, Robinson, et al., Eds. 1-29, 1997).

Following exposure of immune response cells to CG-encoding nucleic acid constructs, the encoded antigens are produced intracellularly and depending on the attached targeting signals, can be directed toward major histocompatability complex (MHC) class I or II presentation, resulting in the stimulation of specific antibodies and/or a CTL-mediated response.

See, e.g., Tang et al., 1992; Cox et al., 1993; Fynan et al., 1993; Ulmer et al., 1993; Wang et al., 1993 and Whitton et al., 1993.

IV. The Immune Response to hCG Peptides and Polypeptides

In experimental systems, tumor antigen specific cytotoxic T lymphocytes (CTL) are the most powerful immunological mechanism for the elimination of tumors. CTL can be induced either in vivo with vaccines or can be generated in vitro and then be re-infused into the tumor-bearing organism. The in vivo induction of CTL is typically accomplished by immunization with live virus or cells (Tanaka, et al., J. Immunol., 147, 3646-52, 1991; Wang, et al., J. Immunol., 4685-4692, 1995; Torre-Amione, et al., Proc. Natl. Acad. Sci. U.S.A., 87:1486-90, 1990.

With the exception of a few special viral proteins such as the SV-40 large T antigen and the hepatitis B surface antigen, injection of isolated or soluble protein antigens does not result in induction of CTL (Schirmbeck et al., Eur. J. Immunol., 23:1528-34, 1993).

A cellular immune response does not normally take place following active immunization with peptide antigens or native proteins such as hCG or peptides derived therefrom.

Administration of a DNA vaccine is expected to elicit the response of both arms of the immune system. The selection of specific vectors may cause one or both types of immune responses to be favored. If the expression of hCG-encoding nucleic acid sequences is modest, then the distribution of the translated peptide will be limited within the cell resulting in antigen presentation in the context of MHC I. If expression of the hCG-encoding nucleic acid sequences is robust then the translated peptide is expected to distribute beyond the confines of the cell and appear in the bloodstream resulting in MHC II-mediated responses as well.

The DNA vaccines of the present invention provide the advantage that following exposure to the immune response cells of the subject, the cells express the processed antigen in association with class I and class II MHC antigens and are thus effective to stimulate a cellular immune response by the subject (e.g., a primary immune response when contacted with naive CTL).

In addition, DNA vaccines provide the ability to prime cells for T helper 1 (Tₕ₁) and T helper 2 (Tₕ₁) biased responses. Tₕ₂ biased responses support the raising of complement-binding subclasses of immunoglobulin G (IgG) and activate phagocytic cells. In contrast, Tₕ₂ biased responses support the raising of immunoglobulin E (IgE), non-complement-binding subclasses of IgG and activate non-phagocytic cells, such as mast cells.

Intramuscular saline injection of DNA vaccines has been successful in eliciting Tₕ₁ biased responses and the injected DNA has been observed to move in the blood to the spleen. Gene gun delivery of DNA vaccines to the epidermis has been successful in eliciting Tₕ₂ biased responses by virtue of transfected epidermal Langerhans cells moving in the lymph to draining nodes. (Robinson, H L and Torres, C A, 1997).

The invention provides hCG DNA vaccine compositions and methods for producing an immune response against hCG in a subject by exposing the immune response cells of the subject to a nucleic acid construct encoding at least one hCG immunogenic epitope or precursor thereof wherein the nucleic acid construct is taken up and processed by such immune response cells.

In some cases, the method further includes detecting the immune response of the subject to one or more expressed hCG immunogenic epitopes.

The cells of the subject may be exposed to the hCG nucleic acid constructs of the present invention by use of a single vector or coding sequence in the primary and subsequent exposure(s) (homologous boosting), while in other cases two different vectors or coding sequences may be used (i.e., heterologous boosting).

In the case of a humoral immune response, the methods of the invention provide for the production of antibodies which specifically bind to hCG. By “specifically bind” herein is meant that the antibodies bind to hCG with a binding constant in the range of at least 10⁶-10⁸ M, with a preferred range being 10⁷-10¹⁰ M.

In the case of a cellular immune response, the methods of the invention provide for expression of immunogenic hCG epitopes together with Class I MHC such that T cell-mediated immune responses may take place.

IV. Preparation of an hCG DNA Vaccine

Various hCG polypeptide chains have been expressed, via recombinant DNA technology, in host cells such as bacteria, yeast, and cultured mammalian cells. See, e.g., Fiddes, J. C. and Goodman, H. M. Nature, vol. 281, pp. 351-356, 1979 and Fiddes, J. C. and Goodman, H. M., Nature, vol. 286, pp. 684-687, 1980, which describe the cloning of the alpha and beta subunits of human chorionic gonadotropin (hCG), respectively.

The construction of bacterial plasmids which serve as DNA vaccines is accomplished using standard recombinant DNA technology. Once constructed, the vaccine plasmid is introduced (transformed) into bacteria. The growth of the bacteria produces many plasmid copies. The plasmid DNA is purified from the bacteria, using relatively simple techniques for separating small circular plasmid DNAs from the much larger bacterial DNA and other bacterial impurities. The purified DNA (a stable molecule) is one type of nucleic acid construct which may be used as a DNA vaccine.

In one embodiment, the nucleic acid construct or DNA vaccines of the invention is a heterologous hCG-encoding nucleic acid construct comprising the polynucleotide sequence, bounded by an initiation site and a termination site, that is transcribed to produce a primary transcript. As used herein, a “heterologous hCG-encoding nucleic acid construct” also referred to as an “hCG-encoding nucleic acid construct” includes at least two components: (1) the DNA encoding one or more hCG immunogenic epitopes, and (2) a transcriptional promoter element or elements operatively linked for expression of the hCG antigen-encoding DNA.

The hCG-encoding nucleic acid constructs of the invention are inserted into a vector which includes sequences for expression of the hCG-encoding genes. Such hCG-encoding nucleic acid constructs can be produced by a number of known methods. All of the techniques used herein are described in detail in Maniatis et al, (1982) MOLECULAR CLONING: A LABORATORY MANUAL (Cold Spring Harbor Laboratory), expressly incorporated by reference herein.

In some cases, the heterologous hCG-encoding nucleic acid construct is carried in any of a number of vectors useful for the transfection of mammalian cells, such as a DNA plasmid vector, into which an hCG-encoding nucleic acid construct has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. Appropriate cloning and expression vectors for use with prokaryotic and eukaryotic hosts are also described in Sambrook et al., 1989, expressly incorporated by reference herein.

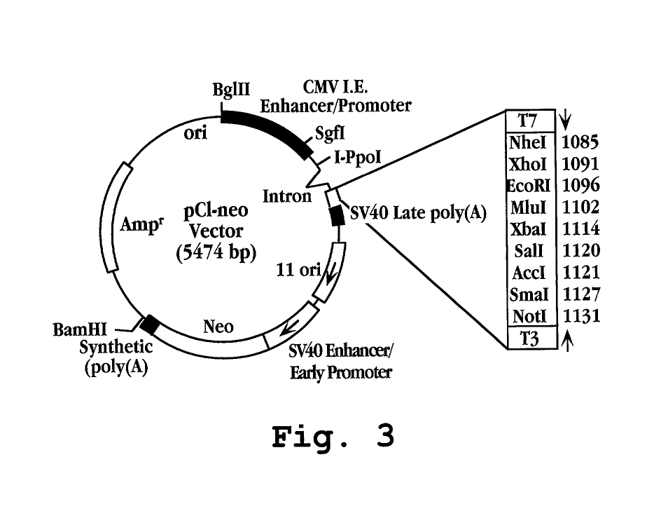

One preferred plasmid vector is the pCI-neo mammalian expression vector (Promega, Madison, Wis., FIG. 3), which can be used to transfect both bacteria and eukaryotic cells. The vector may include any of a number of promoters useful for the expression of mammalian genes. A preferred promoter is the human cytomegalovirus (CMV) immediate early enhancer/promoter region useful to promote constitutive expression of cloned DNA inserts in mammalian cells. The CMV promoter and enhancer has been identified to be active primarily in rapidly dividing cells, since the enhancer is activated by transacting factors present in the nucleus (Ghazal P, et al., Proc Natl Acad Sci U S A 84:3658-62, 1987). The CMV promoter is the strongest identified thus far and as a result, it is useful in DNA vaccine construction (Roth, J. et al., J Natl Cancer Inst 89: 21-39, 1997).

The expression vector can optionally include additional sequences such as enhancer elements, splicing signals, termination and polyadenylation signals, viral replicons, bacterial plasmid sequences, additional restriction enzyme sites, multiple cloning sites, other coding fragments, and sequences effective to reduce degradation of the DNA plasmid vector, i.e., by exonucleases, such that their overall length may vary considerably. It is therefore contemplated that a nucleic acid construct of almost any length may be employed, with the total length determined by the ease of preparation and the intended recombinant DNA protocol.

The hCG-encoding nucleic acid constructs of the invention encompass biologically-functional equivalent peptides. Such sequences may arise as a consequence of codon redundancy and functional equivalency that are known to occur naturally within nucleic acid sequences and the proteins thus encoded. Alternatively, functionally-equivalent proteins or peptides may be created by making changes in the protein structure, based on considerations of the properties of the amino acids being exchanged. Such changes designed may be introduced through the application of site-directed mutagenesis techniques, e.g., to introduce improvements to the immunogenicity of the protein or to test mutants in order to examine activity at the molecular level.

Exemplary additional sequences include, but are not limited to an ATG start codon 5′ to the coding sequence, a TAA stop codon 3′ to the coding sequence, a 5′ cap, a 5′ UTR composed of the multiple cloning site, a short 3′ UTR, a 3′ poly A tail of about 200 nucleotides, an intron 5′ to the hCG coding sequence, and a poly U sequence.

The expression vector may or may not include a selectable marker. Exemplary selectable markers include the genes encoding neomycin phosphotransferase and beta lactamase. However, the presence of beta lactamase or neomycin phosphotransferase in the vector may result in unnecessary immune responses by the subject. Hence, in some cases, removal of these components from the DNA vaccine is desired. The hCG-encoding nucleic acid constructs may be modified in a number of ways typically employed by those of skill in the art, in order to remove these components.

It will be understood that when exposed to the immune response cells of a subject, linear nucleic acid constructs may be susceptible to attack by nucleases, in particular, exonucleases.

In one exemplary modification, stability of such a linear construct can be achieved through use of a chimeric oligonucleotide primer composed of phosphodiamidate morpholine nucleic acids linked synthetically to phosphodiester nucleic acids at the 3′-end to provide resistance to 5′-exonucleases. Such a linear hCG encoding nucleic acid construct may include a chimeric intron which has 3′-phosphodiamidate morpholine (PMO) nucleic acids linked to phosphodiester nucleic acids in the nucleic acid construct. PCR may be used to generate an amplified linear DNA sequence comprising the CMV immediate-early promoter/enhancer, the chimeric intron, the CTP-37 insert flanked by ATG translation start and TAA termination codons, and the SV40 polyadenylation signal. This strategy provides for blunt-ended, double-stranded DNA with exonuclease protection on the 5′ residues of both strands. Exemplary sequences include, but are not limited to, 5′-TCCATGTCGGTCCTGATGCT-3′ (SEQ ID NO:24), a sequence motiff which has been shown to stimulate an immune response when composed of phosphorothioate intemucleoside linkages; and 5′-ACCACCC-3′(SEQ ID NO:25), an antisense Shine-Delgarno sequence considered incapable of interfering with eukaryotic gene expression, which should be sufficient to protect adjacent nuclease sensitive sequences.

Another approach is based on a 5′ untranslated region (UTR) containing a poly-U sequence. This poly-U sequence is designed to form a triple helical cap on the 3′-poly-A portion of the transcript, resulting in circular DNA which is very resistant to nucleolytic attack due to the lack of free ends for exonuclease activity as well as endonuclease activity.

In a further approach, the nucleic acid construct comprises an RNA transcript operably linked to one or more additional RNA sequences effective to express said one or more immunogenic human chorionic gonadotropin (hCG) epitopes or precursors thereof in a subject. Such RNA transcripts are prepared by the use of T7 RNA polymerase to generate an essential mRNA transcript containing the coding sequence, e.g., the CTP 37-mer insert (SEQ ID NO:1), flanked by an ATG translational start codon and TAA. This transcript can be prepared with a 5′-cap, a short 5′-untranslated region (UTR) composed primarily of the multiple cloning region, or a short 3′-UTR and a fragment of approximately 200 adenosine residues. Such transcripts may be prepared by techniques routinely employed by those of skill in the art, and which are commercially available, e.g., the mESSAGE mMACHINE™ (Ambion Inc., Austin, Tex.).

Such a transcript provides the advantage that it does not contain ancillary sequence and is the ultimate precursor to translation. Protection against degradation can be achieved through the use of a poly-T phosphorodiamidate morpholine oligomer which can hybridize to the 3′-end of the transcript and protect from 3′ to 5′ exonuclease activity and does not interfere with translation. The 5′-end of the transcript may not require improved stability due to the 5′ cap or can be stabilized with an antisense oligonucleotide at the AUG translation start site composed of 2′-O-methyl residues which do not efficiently inhibit translation when at the AUG site. Alternatively the 5′-end may be protected via T4 RNA ligase ligation to a 2′-O-methyl oligonucleotide which is resistant to 5′-exonuclease activity.

Once an hCG-encoding nucleic acid construct has been prepared, the construct is verified to contain the correct insert by sequencing from both directions using T7 or T3 primer sites, using techniques well known in the art. The functional verification that the construct encodes an immunogenic hCG epitope may be accomplished by way of in vitro translation. An exemplary method employs a plasmid insert construct in a rabbit reticulocyte lysate and a solid support hCG antigen capture assay (ELISA) using antibodies, specific to a particular hCG peptide, e.g., the CTP-37-mer (SEQ ID NO:1). Appropriate controls are also prepared in order to positively identify constructs with the correct hCG-encoding nucleic acid coding sequence.

The invention provides hCG-encoding nucleic acid constructs described above, and methods of exposing the immune response cells of a human subject to such constructs. Such exposure may take place in vitro or in vivo and the immune response cells of the subject may be exposed to hCG-encoding nucleic acid constructs individually or two or more types of hCG-encoding nucleic acid constructs may be provided to the cells, either at the same time or sequentially. In one preferred embodiment, the DNA vaccine is introduced into dendritic cells, i.e., by ex vivo transfection into DC of a subject, followed by return of the transfected cells to the subject. Intramuscular delivery of DC transfected in vitro with a DNA vaccine encoding two Herpes Simplex Virus (HSV) proteins was shown to enhance resistance to viral challenge (Manickan E et al., J. Leuk. Biol. 61(2):125-132, 1997).

It should be understood that the hCG-encoding nucleic acid construct itself is expressed in the subject's cells using transcription factors provided by the subject, or provided by the hCG-encoding nucleic acid construct.

Exposure of a subject's immune response cells to hCG-encoding nucleic acid constructs encoding one or more hCG immunogenic epitopes may be used to induce strong and long-lived humoral and cellular immunity to such epitopes.

V. hCG Immunogenic Epitopes and Coding Sequences Therefor

An “hCG immunogenic epitope” or “hCG epitope” is any amino acid sequence, or combination of amino acid sequences which elicits an immune response against hCG. The encoded antigens can be hCG peptides or polypeptides. The hCG peptides and polypeptides can be of various lengths, and may undergo normal host cell processing to yield smaller fragments than the initial translation product.

The hCG peptides and polypeptides may also undergo normal host cell post-translational modification such as glycosylation, myristoylation, or phosphorylation. In addition, the expressed hCG peptides and polypeptides may be expressed intracellularly, extracellularly, on the cell-surface or be released from the cells in which they are produced.

The hCG DNA vaccines described herein are representative of the types of DNA vaccines that can be used in the current invention. In general, the size of the encoded polypeptide antigen must be at least large enough to encompass one or more immunogenic epitopes of hCG. The smallest useful immunogenic epitope or fragment anticipated by the present disclosure is generally about 8 contiguous amino acid residues in length, with sequences on the order of about 8 to about 40 or more of amino acids preferred. However, the size of the encoded antigen may be relatively large, for example up to several hundred or more amino acids, so long as expression of the hCG polypeptide results in processing by the host to yield one or more hCG immunogenic epitopes capable of eliciting an immune response to hCG.

It is appreciated that although the active domain on the surface of the polypeptide may comprise a single discrete segment of the primary amino acid sequence of the polypeptide, in many instances, the active domain of a native folded form of a polypeptide comprises two or more discontinuous amino acid segments in the primary amino acid sequence of the parent polypeptide. Moreover, when the sequence of the desired immunogenic epitope (i.e., an hCG peptide) is known, a suitable coding sequence for the polynucleotide can be inferred.

In one preferred embodiment, the hCG-encoding nucleic acid construct comprises the coding sequence for the CTP of hCG (SEQ ID NO:2), alone or in combination with the coding sequence for one or more additional immunogenic hCG epitopes. In another preferred embodiment, the hCG-encoding nucleic acid construct comprises the coding sequence for the CTP (SEQ ID NO:2) and the “loop” peptide (SEQ ID NO:6), alone or together with the coding sequence for one or more additional immunogenic hCG epitopes.

In a further preferred embodiment, the hCG-encoding nucleic acid construct comprises the coding sequence for two or more epitopes taken from the sequence encoding the entire beta subunit of hCG (SEQ ID NO:14), each having at least 8 amino acids, with a linker sequence between them, e.g., the nucleic acid sequence presented as SEQ ID NO:10, which encodes a fusion protein having the sequence presented as SEQ ID NO:5.

Alternatively, a nucleic acid construct of the invention comprises the coding sequence for one or more immunogenic epitopes of the beta subunit of hCG, e.g., SEQ ID NO:3 and/or SEQ ID NO:4. If more than one epitope is included, they may be encoded by a single nucleic acid construct or each epitope may be encoded by a separate hCG-encoding nucleic acid construct with both nucleic acid construct delivered to the subject, either at the same time or at different times.

In some cases, the nucleic acid constructs of the invention comprise the coding sequence for a single immunogenic epitope. In a preferred embodiment, the nucleic acid construct encodes more than one hCG immunogenic epitope. A nucleic acid sequence encoding two or is more peptides may be co-expressed, either individually or as a fusion protein. At least one of the two or more peptides is an hCG immunogenic epitope.

In other cases, the CTP of beta hCG (SEQ ID NO:2) or an epitope thereof, exemplified by the sequences presented as SEQ ID NO:3 and SEQ ID NO:4 is administered to the subject, resulting in a humoral immune response to one or more immunogenic epitopes. About 10 weeks later, this is followed by administration of a DNA vaccine of the invention, as further described herein.

In a further embodiment, a DNA vaccine of the invention comprises a coding sequence for a fused polypeptide with a first sequence encoding an hCG immunogenic peptide or precursor thereof in reading frame alignment with a one or more additional sequences encoding another polypeptide, e.g., a cytokine such as GM-CSF, an immune system stimulator or another cancer-associated antigen, such that administration of the DNA vaccine results in co-expression of the hCG peptide along with the cytokine, immune system stimulator or other cancer- associated antigen in the same host cell.

Immune response modulators for use in the invention include, GM-CSF, IL-4, IL-12 and co-stimulatory molecules such as B7.1 and B7.2. (See, e.g., Robinson, H L and Torres, C A, 1997.) A DNA vaccine encoding murine GM-CSF has been shown to enhance the protective effect to a malaria DNA vaccine in mice (Weiss W R et al., J. Immunol. 161(5)2325-2332, 1998).

Such immune response modulators may be targeted to tumor cells or in the vicinity of a tumor in order to enhance the immune response to the one or more antigens encoded by an hCG DNA vaccine.

Exemplary cancer-associated antigens include the HER-2/neu oncogenic protein and immunogenic epitopes thereof. HER-2/neu-directed monoclonal antibody therapy has been effective in eradicating malignancy in animal models and has shown benefit in the treatment of human HER-2/neu-overexpressing cancers (Disis M L, et al., Adv Cancer Res 71: 343-71, 1997). In one embodiment, the invention provides fusion proteins comprising immunogenic HER-2/neu peptides fused to immunogenic hCG beta subunit peptides, with or without a linker sequence in between the peptides.

Exemplary coding sequences for Her-2/hCG fusion proteins include SEQ ID NO:16 which encodes a fusion protein (SEQ ID NO:11), consisting of the hCG CTP (SEQ ID NO:2), a linker (SEQ ID NO:9), and amino acids 115-136 of the Her-2 protein (LAVLDNGDPLNNTTPVTGASPG, SEQ ID NO:19); SEQ ID NO:17 which encodes a fusion protein (SEQ ID NO:12), consisting of the hCG CTP (SEQ ID NO:2), a linker (SEQ ID NO:9), and amino acids 134-151 of the Her-2 protein (LPGGLRELQLRSLTEILKG, SEQ ID NO:20); and SEQ ID NO:18 which encodes a fusion protein (SEQ ID NO:13), consisting of the hCG CTP (SEQ ID NO:2), a linker (SEQ ID NO:9), and amino acids 376-395 of the Her-2 protein (LFLPESFDGDPASNAPLQPE, SEQ ID NO:21).

In some cases the nucleic acid construct for DNA vaccination comprises RNA encoding one or more CG peptides, polypeptides or precursors thereof.

B. Variant or Modified Immunogenic hCG Peptides

In some cases, the hCG-encoding nucleic acid constructs of the invention encode hCG polypeptides which are presented to the host immune system as fragments of the target protein following initial translation or processing by the host, rather than the intact target protein. In other words, the peptide or polypeptide which is expressed may or may not have the entire amino acid sequence encoded by the hCG-encoding nucleic acid construct.

In other cases, some substitution of amino acids may be possible without effecting the immunogenic character of the fragment. It will also be understood that amino acid and nucleic acid sequences described herein may include additional residues, such as additional N- or C-terminal amino acids or 5′ or 3′ sequences, and yet still be essentially the same as one of the sequences disclosed herein, so long as the peptide or fusion protein maintains the appropriate biological activity, e.g., immunogenicity. The addition of terminal sequences particularly applies to nucleic acid sequences that may, for example, include various non-coding sequences flanking either the 5′ or 3′ portion of the coding region or may include various internal sequences, i.e., introns.

Modifications and changes may be made in the nucleic acid sequence which encodes an hCG peptide, polypeptide, precursor thereof or hCG fusion protein of the invention which still yield a functional molecule that encodes a protein or peptide which is immunogenic. The amino acids of an hCG peptide or polypeptide may be changed in a manner to create an equivalent, or even an improved molecule. The amino acid changes may be achieved by changing the codons of the DNA sequence, such that certain amino acids are substituted for other amino acids in the hCG peptide or polypeptide with improved, or at least without appreciable loss of immunogenicity. It is the immunogenicity of the hCG peptide or polypeptide which defines its biological activity. Certain amino acid sequence substitutions can be made in the amino acid sequence, and the nucleic acid sequence encoding it, which result in expression of an hCG peptide or polypeptide with greater biological utility or activity. In some case such changes may provide other benefits, e.g., stability or more desirable pharmacologic properties without appreciable loss of biological utility or activity.

In making such changes, the hydropathic index of amino acids may be considered. Certain amino acids may be substituted by other amino acids having a similar hydropathic index or score and still obtain a biological functionally equivalent protein. (Kyte and Doolittle, 1982).

In making such changes, the substitution of amino acids whose hydropathic indices are within +/−2 is preferred, those which are within +/−1 are particularly preferred, and those within +/−0.5 are even more particularly preferred. It is also understood in the art that the substitution of like amino acids can be made effectively on the basis of hydrophilicity. U.S. Pat. No. 4,554,101. In such changes, the substitution of amino acids whose hydrophilicity values are within +/−2 is preferred, those which are within +/−1 are particularly preferred, and those within +/−0.5 are even more particularly preferred.

Site-specific mutagenesis and other techniques, e.g., treatment with mutagenic agents are useful for the preparation of biologically functional equivalent proteins or peptides, through specific mutagenesis of the underlying DNA. Site-specific mutagenesis technique allows for introduction of one or more nucleotide sequence changes into the DNA. In general, the technique of site-specific mutagenesis is well known in the art, as exemplified by various publications and the availability of commercial kits, e.g., QuickChange™ (Stratagene, LaJolla, Calif.).

C. Expression Libraries

The invention also provides methods for eliciting an immune response to a number of hCG immunogenic determinants. A library is constructed by partially digesting the entire coding sequence of the beta subunit of hCG (SEQ ID NO:14) in a manner effective to produce fragments having at least 24 nucleotides (i.e., encoding peptides of at least 8 amino acids).

The genomic or cDNA (or RNA) sample is segmented by physical fragmentation or, preferably, by enzymatic cleavage, using restriction endonucleases to produce relatively small fragments, on the order of about 100 to 1,000 base pairs. In one application of the method, the average fragment size in each plasmid is at least 24 or 30 to about 100 nucleotides. Fragmentation methods are well known to those skilled in the art and may be varied to obtain different mixtures of fragments, i.e., by use of different restriction endonucleases or combinations and digestion times. The construct may or may not contain a signal sequence allowing hCG polypeptides to be secreted. In one preferred embodiment, the average fragment size in each plasmid is on the order of about 100 nucleotides.

After fragmentation of the DNA, an expression library is prepared. Preparation of such libraries is relatively straightforward and can be performed using published methods. The fragmented DNA is incorporated into a standard cloning vector, placed under control of a promoter, bacteria are transformed with the vector, transformants identified and the plasmid DNA isolated using methods known to those of skill in the art.

Once a DNA plasmid library has been obtained, a host is inoculated with a DNA vaccine containing portions of the plasmid library. The library may be inoculated into the host by any one of several different methods known to be effective for DNA vaccination, as summarized below.

After a DNA vaccine consisting of one or more library clones is introduced into the host by of several different methods known to be effective for DNA vaccination, the immune response to the vaccine is evaluated.

D. Evaluation of the Immune Response to a DNA Vaccine

Such analysis includes an evaluation of both the humoral response measured by a determination of antibody production by the host and the cellular immune response by the host measured by a standard CTL assay. Additional assays include measurement of complement activation by evaluating subject blood samples for complement “split products” and evaluating the antitumor effect of a subject's serum on cultured tumor cells.

Analysis of the level of anti-hCG antibody production and hCG levels in the plasma of the host before and after administration of the hCG expression library DNA vaccine may then be used to identify an individual plasmid or combinations of plasmids that are responsible for the immune response. It is then possible to prepare DNA vaccines from the plasmids, and to identify the polypeptides encoded by the plasmid vectors which have been shown to be protective.

The use of the expression library methods of the present invention employ techniques similar to those described by Ulmer et al. 1993, incorporated herein by reference herein. An expression library composed of DNA fragments can be used in virtually any form, including naked DNA and plasmid DNA, and may be administered to the subject in a variety of ways, including mucosal and gene gun inoculation or needle injection into muscle or skin or by oral (parenteral) administration, as described, for example, by Fynan et al. (1993) and Tang et al (1992).

VI. Administering an hCG DNA Vaccine to a Subject

In the methods of the invention, the immune response cells of the subject are exposed to hCG-encoding nucleic acid constructs. Such exposure may take place in vitro and in vivo.

In vivo administration may include delivery of the DNA vaccine in the presence of an adjuvant or other substances that has the capability of promoting nucleic acid uptake or recruiting immune system cells to the site of the inoculation.

In the methods of the present invention, the immune response cells of a subject in whom an immune response is desired, are exposed to a DNA vaccine. Immunization can be achieved following exposure of the immune response cells of the subject to a DNA vaccine encoding an hCG antigen in the form of a peptide, polypeptide precursor thereof or fusion protein. Effective DNA vaccination results in a reduction in the amount of circulating hCG or cell surface hCG in the subject relative to the level of hCG which was detected in the subject prior to administration of the vaccine.

When exposure takes place in vivo, the DNA vaccines of the invention are combined with a physiologically acceptable carrier prior to in vivo administration to the subject. The DNA vaccine may be administered by routes including, but not limited to, inhalation, intradermal (ID) injection, intramuscular (IM) injection, intravenous (IV) injection, intraperitoneal (IP) injection, subcutaneous (SC) injection, peritumoral injection, application to mucosal surfaces (e.g., application of DNA drops to the nares or trachea), intraocular administration, or particle bombardment of the epidermis using a gene gun. (See, e.g., Fynan et al., 1993).

DNA vaccines can be injected in saline solutions into muscle or skin using a syringe and needle (Wolff J A, et al., Science 247:1465 1468, 1990; Raz E, et al., Proc. Natl. Acad. Sci. USA 91:9519-9523, 1994). DNA vaccines can also be administered by coating the nucleic acid onto microscopic gold beads and then using a gene gun to deliver the beads into cells. (See, e.g., Johnston S A and Tang D C, Genet Eng (N Y) 15:225-36, 1993.) The saline injections deliver the nucleic acid into extracellular spaces, whereas gene guns deliver nucleic acid coated gold beads directly into cells. The immune responses raised by injection and bombardment require different amounts of nucleic acid and can result in production of different types of T cells.

Muscle may be conveniently accessed by direct injection through the skin and accordingly is a useful site for the delivery and expression of hCG-encoding nucleic acid constructs. The DNA vaccine can be delivered into muscle by multiple and/or repetitive injections. In this way therapy can be extended over long periods of time.

When muscle cells are injected with hCG-encoding nucleic acid constructs, a selected immune response against the immunogenic polypeptides results following antigen presentation in the context of MHC molecules to provoke both a cellular and humoral immune response specific to the hCG peptides or polypeptides. (See, e.g., Felgner, U.S. Pat. No. 5,703,055) DNA vaccines may also be delivered and their gene products expressed in the epidermis, which is conveniently accessed by direct injection, particle bombardment or electroporation. The DNA vaccine can be delivered into the epidermis by multiple and/or repetitive administrations. In this way therapy can be extended over a long period of time. Skin contains Langerhans cells which function as antigen presenting cells and can present the hCG immunogenic peptides or polypeptides in the context of MHC molecules to provoke both a cellular and humoral immune response specific to the hCG peptides or polypeptides (Felgner, 1998). DNA vaccines delivered into the dermis by electroporation have been shown to prime CTL in vivo (Nomura M et al., 1996).

Peritumoral injection, which limits the administration to a neighboring tumor, and compartmental administration, such as intraperitoneal injection, have both shown efficacy in preclinical models (Wills K N, et al. Hum Gene Ther 5:1079-88, 1994).

DNA vaccines may also be delivered to a mucosal tissue by a variety of methods including nucleic acid-containing nose-drops, inhalants, suppositories, microsphere encapsulated nucleic acids, or by bombardment with DNA coated gold particles. For example, the DNA vaccine can be administered to a respiratory mucosal surface, by way of a gene gun, or by administration of liposomes which contain the DNA vaccine (Fynan et al., 1993; Gregoriadis G et al., FEBS Lett., 402:107-110, 1997). In some cases, the DNA vaccine is introduced by directly inoculating bacteria bearing an hCG-encoding nucleic acid construct into the host or by using another type of infectious agent as a vector, e.g., adenovirus. Use of an orally delivered live attenuated Salmonella bacteria as a carrier for a DNA vaccine vector has been shown to result in effective targeting of in vivo antigen expression to antigen presenting cells (APCs; Paglia P et al., Blood, 92(9):3172-3176, 1998).

### Targeting Tumor Cells

Tumor cells may be targeted by a DNA vaccine, either alone or in combination with a DNA vaccine aimed at immune response cells cells. In one general embodiment, tumor cells are targeted with a viral or other suitable tumor-specific vector carrying a gene for an hCG anitegen, ie teh CTP peptide antigen or an epitope thereof, for increased expression of one or more CTP epitopes on the tumor cells, as a target for immune response. Viral vector systems for use in expressing tumor-specific antigens have been described, e.g., U.S. Pat. No. 5,744,133, and Lan K H; et al., Gastroenterology, 111 (5):1241-51, 1996.

In another general embodiment, carried out in conjunction with either peptide antigen vaccination with the CTP peptide (or an epitoe thereof) or vaccination with a CTP-DNA vaccine, the tumor cells are targeted with a viral or other suitable tumor-specific vector capable of transfecting tumor cells, and expressing an immune stimulator, such as IL-2, IL-4, or GM-CSF in the transfected cells. The secretion of an immune stimulator in the region of the tumor cells is effective to enhance the cytotoxic T cell response against the hCG antigen expressed on the surface of tumor cells.

A variety of viral vectors are available for tumor targeting. Parvivirus are known to infect tumor cells selectively. Alternatively, the virus can be designed to replicate selectively in tumor cells, according to published methods. (See, for example, Puhlmann M; et al., Hum Gene Ther, 10 (4):649-57, 1999; Noguiez-Hellin P; et al. Proc Natl Acad Sci USA, 93(9):4175-80, 1996; and Cooper M J, Semin Oncol 23(1) p172-87, 1996. For example, the virus may be altered to contain a mutated thymidine kinase or polymerase gene that allows viral replication only in rapidly dividing cells containing these enzymes. Alternatively, the virus can be genetically engineered to contain tumor-specific control elements, e.g., tumor-specific promoter regions, that are responsive and express the desired protein or protein necessary for viral replication only in tumor cells.

In one preferred embodiment, administration of a DNA vaccine is accomplished by particle bombardment using gold beads coated with a DNA vaccine of the invention. Preferably, the gold beads are 1 μm to 2 μm in diameter. The coated beads are generally administered intradermally, intramuscularly, directly in the vicinity of the target organ, or by other routes useful in particle bombardment and known to those of ordinary skill in the art. (See, e.g., Robinson, H L and Torres, C A, Sem. Immunol. 9:271-282, 1997.)

DNA vaccines may be delivered in any physiologically compatible medium. For example, saline for injection and gold particles for particle bombardment are suitable for introducing a DNA vaccine into a subject. The hCG-encoding nucleic acid construct may be mixed with excipients which are pharmaceutically acceptable and compatible with the active ingredient. Suitable excipients are, for example, water, saline, dextrose, glycerol, ethanol, or the like and combinations thereof. In addition, if desired, the DNA vaccine may contain minor amounts of wetting or emulsifying agents, pH buffering agents, or adjuvants which can enhance the effectiveness of the vaccines.

The DNA vaccines are administered in a manner compatible with the dosage formulation, and in such amount as will be result in the production of immunogenic peptides. The quantity to be administered depends on the subject to be treated, including, e.g., the capacity of the individual's immune system to respond, and the degree of immunization desired. Accordingly, the amount of the DNA vaccine will vary for cancer therapeutic applications relative to fertility control applications.

The dosage of the vaccine also depends on the route of administration and the size of the host. Successful IM and ID injection of DNA vaccines which introduce the vaccine outside of cells have included from about 1 to 100 μg DNA in mice, while successful gene gun delivery methods have included from about long to 10 μg DNA in mice. Effective dose ranges have been similar for mice, calves and monkeys. Suitable regimes for initial and subsequent administration are also variable, but are typified by an initial administration followed by subsequent inoculations or other administrations. (See, e.g., Robinson, H L and Torres, 1997.) In the systemic DNA vaccine administration strategies presented herein, an effective vaccine dosage will generally be in the range of about 1 μg/kg to about 50 μg/kg of DNA, preferably about 10-25 μg/kg of body weight of the subject. The amount of DNA effective to produce an immune response ranges from between about 0.1 or 1 ng to 50 or 100 μg. However, as will be appreciated, this dosage will vary in a manner apparent to those of ordinary skill in the art according to the particular DNA used, the particular peptide or polypeptide encoded by the DNA, and the subject being inoculated. Likewise, vaccine compositions may vary widely, to include, for example various adjuvants, e.g., muranyl dipeptide or coadministration of a variety of cytokines such as interleukin 1 or interleukin 12.

In the DNA vaccination methods of the present invention, the site of exposure to the nucleic acid construct can be controlled allowing cells not normally exposed to hCG to present hCG antigens.

In some cases, the DNA vaccine is administered to a human subject who has cancer. In such cases, the DNA vaccine contains a nucleotide sequence capable of expressing an immunogenic hCG peptide or an immunogenic fragment thereof, wherein the hCG peptide or fragment has an epitope in common with the hCG antigenic epitope released by the cancer cells of the subject.

Surgery, radiation therapy, and chemotherapy are currently the primary methods for cancer treatment. DNA vaccine based therapies may interact in synergistic or additive ways with them. In some cases, improved methods for treating cancer will combine conventional cancer treatments, e.g., chemotherapy with the administration of hCG-encoding DNA vaccines resulting in a greater therapeutic effect.

In other cases, the DNA vaccine is administered to a mammalian subject as a means of fertility control. In such cases, the DNA vaccine contains a nucleotide sequence capable of expressing an immunogenic hCG peptide or an immunogenic fragment thereof, wherein the immune response to the hCG peptide(s) modulates the level of circulating CG or provides a cell mediated immune response against the placenta and/or blastocyst.

Such hCG DNA vaccine-mediated fertility control is applicable to mammals in general, and has utility in treatment of both humans and animals. For example, DNA vaccines may be administered intradermally to cattle or other livestock animals using a needleless jet injector. (See, e.g., WO 98/03196.)

VII. Genetic (DNA) Vaccination of Mice with hCG-DNA Construct

The adult mouse model is a convenient vehicle in which to determine the immune response to a DNA vaccine.

Mice immunized with an expression construct encoding the intact beta subunit of hCG have been demonstrated to develop both a humoral and cellular immune response to human CG. When such mice were primed by DNA vaccination prior to injection with hCG-expressing SP2/O myeloma cells, a marked reduction in the size of tumors was demonstrated (Geissler M, et al., Lab Invest 76(6):859-71, 1997).

In general, the humoral and cellular immune response to a nucleic acid construct or DNA vaccine may be evaluated in mice which have been inoculated with various amounts of an hCG-encoding nucleic acid construct. Following exposure of immune response cells to the nucleic acid construct, mice may be tested for the presence of antibodies against various immunogenic epitopes of hCG as determined by an in vitro binding assay, such as radioimmunoassay (RIA), enzyme-linked immunoabsorbent assay (ELISA) or immunoprecipitation. Such techniques and assays are known in the art. The cell mediated immune response is also evaluated by culture of splenocytes from the exposed mice and a determination of lysis of ⁵¹Cr-labeled hCG epitope-specific target cells in a standard cell mediated cytotoxicity assay [Wunderlich and Shearer, in Coligan et al. (eds.) CURRENT PROTOCOLS IN IMMUNOLOGY Wiley, New York, N.Y., 1:3.11, 1998].

An exemplary determination of the effectiveness of a DNA vaccine of the invention is carried out as follows. hCG-encoding and control nucleic acid constructs are injected intradermally in the skin of the abdomen or intramuscularly in the quadriceps as a 100 μl solution (1 μg/μl of plasmid in phosphate buffered saline) into C57BL/6 mice or Abgenix xenomice. The injections is repeated at a two week interval. In addition, Diptheria Toxin (DT)-conjugated CTP-37 in Freunds adjuvant is injected into mice in parallel as a positive control.

Antigen specific humoral immunity to hCG and CTP-37 is then evaluated by recovery and analysis of serum from vaccinated mice. The serum is added to hCG or CTP-37 antigen in a solid-support capture immunoassay.

Cell mediated immunity is measured by recovery of spleen cells from vaccinated mice, which are restimulated with hCG for 7 days in the presence of exogenous IL-2, and the cultures evaluated for the presence of antigen specific CTL. The following examples illustrate but are not intended in any way to limit the invention.

### EXAMPLE 1

### Preparation of hCG-encoding Nucleic Acid Constructs

A. DNA Plasmid Vector

The pCI-neo mammalian expression vector purchased from Promega (Madison, Wis., FIG. 3) was double digested with Nhe I (recognition sequence - G-CTAGC) and Eco RI (recognition sequence—G-AATTC) restriction endonucleases to provide incompatible sticky ends [Promega cat # R6501 and R6011 respectively]. The vector also contains the neomycin phosphotransferase gene, a selectable marker for mammalian cells. A descriptive map of the vector includes:

| Functional region | Nucleotides |
|---|---|
| CMV immediate early enhancer | 1-659 |
| CMV immediate early promoter | 669-750 |
| chimeric intron [prevent cryptic 5′splice | 890-1022 |
| T7 promoter [synthesis of RNA in vitro] | 1067-1085 |
| multiple cloning site [insert site] | 1085-1137 |
| SV40 late polyadenylation signal | 1067-1388 |
| [terminates transcription adds poly A] | 1438-1938 |
| f1 origin of replication [high copy number in bacteria] | |
| SV40 enhancer/promoter | 2002-2420 |
| SV40 origin of replication [eukaryotic replication] | 2318-2383 |
| neomycin phosphotransferase | 2465-3259 |
| synthetic polyadenylation signal | 3323-3371 |
| beta lactamase [ampicillin resistance gene] | 3782-4642 |

B. hCG-Encoding Nucleic Acid Constructs

The vector was double digested with Nhe I (recognition sequence—G-CTAGC) and Eco RI (recognition sequence—G-AATTC) restriction endonucleases to provide incompatible sticky ends (Promega). The NheI/EcoRI prepared vector was then combined with the oligonucleotides followed by ligation and bacterial transformation. A variety of inserts were added to allow multiple epitopes to be expressed from either the hCG or other tumor associated antigens.

hCG coding sequences which are added to the vector include the coding sequence for the CTP of the beta subunit of hCG with an added methionine at the N-terminus (SEQ ID NO:7), the coding sequence for the “loop” peptide of the beta subunit of hCG (SEQ ID NO:8) and the coding sequences for two epitopes of the beta subunit of hCG inserted individually into the vector as (SEQ ID NO:22) and (SEQ ID NO:23), respectively, or in a single insert with a linker (SEQ ID NO:10), between the two epitopes. Various exemplary sequences are presented in Table 1.

Sequences which encode Her-2/hCG fusion proteins consisting of the hCG CTP, a linker and an Her-2 peptide are also added to the vector, including SEQ ID NO:16 which encodes amino acids 115-136 of the Her-2 protein, SEQ ID NO:17 which encodes amino acids 134-151 of the Her-2 protein SEQ ID NO:18 which encodes amino acids 376-395 of the Her-2 protein.

PCR is used to generate a linear DNA sequence comprising the amplification of the plasmid region. For example, nucleotides 1 to 1511, including 1388 nucleotides of the plasmid and an insert of about 123 nucleotides containing the CMV immediate-early promoter/enhancer, the chimeric intron, the CTP-37 insert flanked by ATG translation start and TAA termination codons, and the SV40 polyadenylation signal is sequenced using DNA primers and PCR.

In another exemplary approach, T7 RNA polymerase is used to generate the essential mRNA transcript containing the CTP-37 insert flanked by ATG translation start and TAA termination codons. This transcript may be prepared with a 5′-cap, a short 5′-untranslated region (UTR) composed primarily of the multiple cloning region, a short 3′-UTR and a fragment of approximately 200 adenosine residues. Stability is achieved through the use of a poly-T phosphorodiamdate morpholine oligomer which can hybridize to the 3′-end of the transcript and protect from 3′ to 5′ exonuclease activity but will not interfere with translation. In some cases, the 5′-end of the transcript is protected via T4 RNA ligase ligation to a 2′-O-methyl oligonucleotide which is resistant to 5′-exonuclease activity.

All publications, patents and patent applications are herein expressly incorporated by reference in their entirety to the same extent as if each individual publication, patent or patent application was specifically and individually indicated to be incorporated by reference in its entirety.

TABLE 1
DESCRIPTION OF SEQUENCES

| Description | SEQ ID NO | FIG. |
|---|---|---|
| CTP 37-mer peptide TCDDPRFQDSSSSKAPPPSLPSPSRLPGPSDTPILPQ | 1 | 1B |
| (residues 109-145 of beta subunit of hCG) | | |
| CTP 38-mer peptide MTCDDPRFQDSSSSKAPPPSLPSPSRLPGPSDTPILPQ | 2 | 1C |
| (residues 109-145 of beta subunit of hCG + N-terminal met) | | |
| epitope #1 MDDPRFQDS (8mer peptide corresponding to residues 111-118 of | 3 | 1D |
| beta subunit of hCG, with an added N-terminal methionine) | | |
| epitope #2 RLPGPSDTPILP | 4 | 1E |
| (12mer peptide, residues 133-144 of beta subunit of hCG) | | |
| epitope #1, linker, epitope #2 (peptide) | 5 | 1G |
| DDPRFQDSILPQLLLDRLPGPSDTPILP | | |
| Loop peptide CTP MTR VLQ GVL PAL PQV VC (res 38-57 of beta subunit | 6 | 1H |
| with an S—S bond between residues 38 and 57) | | |
| Insert #1 dsDNA encoding CTP 38 mer | 7 | 2B |
| Insert #2 dsDNA encoding loop | 8 | 2C |
| Linker peptide ILPQLLLD | 9 | 1F |
| DNA encoding the linker peptide designated as SEQ ID NO:9 | 10 | 2D |
| Insert #2-1 (peptide) CTP 38-mer, linker, Her 2 115-136 | 11 | 1I |
| MTCDDPRFQDSSSSKAPPPSLPSPSRLPGPSDTPILPQILPQLLLDLAVLDN | | |
| GDPLNNTTPVTGASPG | | |
| Insert #2-2 (peptide) CTP 38-mer, linker, Her 2 134-151 | 12 | 1J |
| MTCDDPRFQDSSSSKAPPPSLPSPSRLPGPSDTPILPQILPQLLLDLPGGLR | | |
| ELQLRSLTEILKG | | |
| Insert #2-3 (peptide) CTP 38-mer, linker, Her 2 376-395 | 13 | 1K |
| MTCDDPRFQDSSSSKAPPPSLPSPSRLPGPSDTPILPQILPQLLLDLFLPESF | | |
| DGDPASNAPLQPE | | |
| hCG beta sub unit amino acid sequence-full length | 14 | 1A |
| GenBank Accession No. J00117-hCG beta subunit mRNA, complete cds | 15 | 2A |
| DNA encoding Her 2 amino acids 115-136 | 16 | 2E |
| DNA encoding Her 2 amino acids 134-151 | 17 | 2F |
| DNA encoding Her 2 amino acids 376-395 | 18 | 2G |
| amino acids 115-136 of the Her-2 protein | 19 | 1L |
| LAVLDNGDPLNNTTPVTGASPG | | |
| amino acids 134-151 of the Her-2 protein | 20 | 1M |
| LPGGLRELQLRSLTEILKG | | |
| amino acids 376-395 of the Her-2 protein | 21 | 1N |
| LFLPESFDGDPASNAPLQPE | | |
| DNA encoding epitope #1 | 22 | 2A |
| GATGACCCCCGCTTCCAGGACTCC- | | |
| DNA encoding epitope #2 | 23 | 2A |
| CGACTCCCGGGGCCCTCGGACACCCCGATCCTCCCA | | |
| TCCATGTCGGTCCTGATGCT (in text) | 24 | N/A |
| ACCACCC (in text) | 25 | N/A |

What is claimed is:

1. A method for inducing an immune response to hCG in a mammalian subject comprising:
exposing immune response cells of the subject to a nucleic acid construct encoding at least one hCG immunogenic epitope, precursor or fusion protein thereof wherein said nucleic acid construct is taken up and processed by such immune response cells.

2. The method according to claim 1, wherein said exposing takes place in vivo.

3. The method according to claim 2, wherein said exposing is accomplished by introducing said nucleic acid into a subject in a manner effective to induce, cell-surface presentation of one or more hCG peptide antigens against which an immune response is desired.

4. The method according to claim 1, wherein said exposing takes place in vitro.

5. The method according to claim 4, wherein said exposing is accomplished by adding said nucleic acid construct to the cells of a subject in vitro in a manner effective to induce in the cells, cell-surface presentation of one or more hCG peptide antigens against which an immune response is desired, and readministering the exposed cells to the subject.

6. The method according to claim 1, wherein said exposing is effective to induce a cellular immune response against one or more hCG immunogenic epitopes in the subject.

7. The method according to claim 1, wherein said exposing is effective to induce a humoral immune response to one or more hCG immunogenic epitopes in the subject.

8. The method according to claim 1, wherein said nucleic acid construct is a DNA plasmid vector.

9. The method according to claim 1 wherein said nucleic acid construct comprises an RNA transcript operably linked to one or more additional RNA sequences effective to express said one or more immunogenic human chorionic gonadotropin (hCG) epitopes or precursors thereof in a subject.

10. The method according to claim 1, wherein said nucleic acid construct encodes a peptide having at least 8 amino acids taken from the beta subunit of hCG (SEQ ID NO:14).

11. The method according to claim 1, wherein said nucleic acid construct encodes at least one hCG immunogenic epitope selected from the group consisting of amino acids 109 to 145 (SEQ ID NO:1), amino acids 111 to 118 (SEQ ID NO:3), amino acids 133 to 144 (SEQ ID NO:4), and amino acids 38 to 57 (SEQ ID NO:6) of the beta subunit of hCG (SEQ ID NO:14).

12. The method according to claim 1, comprising an hCG fusion protein selected from the group consisting of SEQ ID NO:5, SEQ ID NO:11, SEQ ID NO:12, and SEQ ID NO:13.

13. The method according to claim 1, wherein said mammalian subject is a human cancer patient wherein hCG is associated with a type of cancer selected from the group consisting of colorectal cancer, breast cancer, and lung cancer.

14. The method according to claim 1 wherein said at least one hCG immunogenic epitope includes the peptide presented as SEQ ID NO:2 together with one or more additional hCG peptides.

15. The method according to claim 1 wherein said mammalian subject is a human or animal and said immune response to hCG is effective for fertility control.

16. The method according to claim 1 wherein said at least one hCG immunogenic epitope includes at least two peptides having an amino acid sequence taken from amino acids 109 to 145 of the beta subunit of hCG (SEQ ID NO:1), each peptide having at least 8 amino acids.

17. The method according to claim 1 wherein said at least one hCG immunogenic epitope includes the peptides having sequences presented as (SEQ ID NO:3), and (SEQ ID NO:4).

18. The method according to claim 1 wherein said nucleic acid construct comprises control sequences selected from the group consisting of an ATG start codon 5′ to the coding sequence, a TAA stop codon 3′ to the coding sequence, a 5′ cap, a 5′ UTR composed of the multiple cloning site, a short 3′ UTR and a 3′ poly A tail of about 200 nucleotides.

19. The method according to claim 8 wherein said DNA plasmid vector comprises a sequence having an intron 5′ to the hCG coding sequence, said intron effective to reduce degradation of the DNA plasmid vector.

20. The method according to claim 19 wherein said intron is a chimeric intron having phosphodiamidate morpholine (PMO) nucleic acids linked to phosphodiester nucleic acids.

21. A method for treating cancer in a human subject comprising:
(a) fragmenting the DNA sequence which encodes the beta subunit of hCG (SEQ ID NO:14) in a manner effective to produce fragments having at least 24 nucleotides;
(b) incorporating the fragments into one or more expression vectors capable of being taken up and processed by the immune response cells of the subject;
(c) cloning the expression vectors and producing the cloned expression vectors; and
(c) exposing said immune response cells to said cloned expression vectors in an amount sufficient to vaccinate the immune response cells.

22. The method according to claim 21 further comprising:
(d) analyzing the plasma and/or cells of said subject for an immune response against hCG.

23. A nucleic acid construct for use in a DNA vaccine, comprising the coding sequence for a fusion protein having at least one hCG immunogenic epitope, in a pharmaceutically acceptable carrier.

24. The nucleic acid construct according to claim 23, wherein said at least one hCG immunogenic epitope includes the peptides presented as SEQ ID NO:2 and SEQ ID NO:6.

25. The nucleic acid construct according to claim 23, which encodes at least one hCG immunogenic epitope comprising two or more peptides taken from amino acids 109 to 145 of the beta subunit of hCG (SEQ ID NO:14), each epitope having at least 8 amino acids.

26. The nucleic acid construct according to claim 23, wherein said construct is a DNA plasmid vector.

27. The nucleic acid construct according to claim 26, wherein the sequence of said DNA plasmid vector comprises an intron 5′ to the hCG coding sequence, said intron effective to reduce degradation of said DNA plasmid vector.

28. The nucleic acid construct according to claim 27, wherein said intron is a chimeric intron which comprises phosphodiamidate morpholine (PMO) nucleic acids linked synthetically to phosphodiester nucleic acids.

29. A DNA vaccine cell composition comprising:
immune response cells of a subject prepared by exposing said cells to a nucleic acid construct encoding at least one hCG immunogenic epitope or precursor thereof, wherein said nucleic acid construct is taken up and processed by such immune response cells.

30. The method according to claim 29, wherein said immune response cells provide cell surface presentation of an hCG immunogenic peptide against which an immune response is desired.
